(19) United States
(12) Patent Application Publication
(10) Pub. No.: US 2005/0049208 A1
(43) Pub. Date: Mar. 3, 2005

(54) Method of treating and method of preventing diabetes

(76) Inventors: Doug A. Kaufmann, Rockwall, TX (US); David A. Holland, Arlington, TX (US)

Correspondence Address: Michael A. O'Neil;Michael A. O'Neil, P.C., Suite 820, 5949 Sherry Lane, Dallas, TX, 75225, US

(21) Appl. No.: 10/798,691

(22) Filed: Mar. 11, 2004

Related U.S. Application Data
(63) Continuation-in-part of application No. 10/674,145, filed on Sep. 29, 2003.
(60) Provisional application No. 60/499,976, filed on Sep. 3, 2003.

(51) Int. Cl. A61K 31/7048; A61K 31/496; A61K 31/4196; A61K 31/4178

(52) U.S. Cl. 514/28; 514/254.07; 514/383; 514/397; 424/439

(57) ABSTRACT

This invention comprises a method of treating diabetes. Specifically, the method involves administering one or more anti-fungal agents in amounts, at frequencies, and for durations which are effective in preventing and treating diabetes. The method further comprises the administration of a low carbohydrate diet which may be used either in combination with the aforesaid anti-fungal agent or separately therefrom.

### CROSS-REFERENCE TO RELATED APPLICATION

This application is a continuation-in-part of application Ser. No. 10/674,145 filed Sep. 29, 2003, currently pending, which claims priority based on provisional patent application Ser. No. 60/499,976 filed Sep. 3, 2003.

### TECHNICAL FIELD

This invention relates generally to the prevention and treatment of diabetes, and more particularly to a method for the prevention and a method for the treatment of diabetes utilizing anti-fungal agents and/or anti-fungal dietary regimes.

### BACKGROUND AND SUMMARY OF THE INVENTION

As used herein the term “diabetes” means both type 1 diabetes and type 2 diabetes. Type 1 diabetes, also known as juvenile-onset diabetes, describes the condition wherein an animal or human displays abnormally high levels of serum glucose due to lack of endogenously-produced insulin. The lack of insulin in type 1 diabetes is brought about by the destruction of the insulin-producing beta cells in the islets of Langerhans in the pancreas. Blood sugar control in type 1 diabetes therefore requires life-long use of exogenous insulin, whether in the form of self-administered injections or automatic insulin-infusing pumps, or an islet-cell transplantation procedure. The cause of type 1 diabetes is unknown at this time, although there is speculation about genetic, auto-immune, infectious, or environmental causes. On the other hand, there are specific, known causes of type 1 diabetes, such as certain chemotherapeutic drugs, such as streptozotocin, and other, fungal metabolites, known as mycotoxins, where the prefix “myco” means fungus.

Type 2 diabetes, formerly called adult-onset diabetes, is not as much characterized by the lack of insulin but rather the resistance of the peripheral tissues to the action of insulin. In this case, pancreatic cells are typically producing normal or near-normal amounts of insulin, but the cells elsewhere in the body (for example, muscle cells), have become resistant to insulin's action of causing the uptake of glucose from the bloodstream into the cell. Therefore, high levels of serum glucose are also a result of type 2 diabetes. The cause of type 2 diabetes is also currently unknown, but there have been strong correlations with an unhealthy diet and sedentary lifestyle. Type 2 diabetes represents 90-95% of all cases of diabetes in America.

Diabetes of either form is associated with long-term complications, known as co-morbidities, such as heart, eye and kidney disease, and peripheral blood vessel disease, which can lead to nerve damage and poor wound healing, which can in turn lead to amputation of extremities. People with diabetes are encouraged to actively participate in the management of their diabetes, and maintain good control of their blood sugar in order to minimize the number and severity of such complications. Yet the American Diabetes Association asserts that, despite adequate blood sugar control with either insulin or oral, prescriptive, blood sugar-lowering medications, up to one third of all people with diabetes will still suffer from these secondary, long-term effects of diabetes. This implies that there is an outside factor—something other than sugar or blood sugar alone—that is contributing to the ravages caused by diabetes.

Advances in diabetes treatment techniques, including chemotherapeutical, pharmaceutical, surgical, immune-modulating, and vaccine-related techniques, are well publicized. Unfortunately, none of these techniques is useful until the existence of diabetes has been confirmed. Equally unfortunate is the fact that no technique currently exists for treating diabetes-like symptoms prior to confirmation that diabetes does in fact exist.

The present invention comprises the use of antifungals—medicinal, synthetic, and naturally-occurring—in the prevention and treatment of diabetes in mammals. The use of the antifungal substances pertains to preventive use, empiric use, and specifically-directed use of the antifungals toward the treatment and prevention of diabetes in mammals. Preventive use means using antifungal substances to avoid instances of diabetes altogether. Empiric use indicates the use of an antifungal substance when the onset of diabetes is suspected. Specifically-directed use applies when diabetes has been confirmed by laboratory tests such as serum or urine glucose levels, c-peptide levels, Hemoglobin Al-c levels, or has been confirmed when a related disease commonly found in diabetes is diagnosed and the diagnosis of the related disease leads to a search for and the confirmation of the diagnosis of diabetes.

The present invention also comprises the use of a specific diet for both the treatment and the prevention of diabetes. A specific diet is used in conjunction with or apart from antifungal medications or naturally-occurring antifungal substances. The diet is a low-carbohydrate type of diet that is designed to be low in sugars in the form of simple and complex carbohydrates, and therefore low in naturally-occurring, disease-causing, and immune-suppressing fungal toxins, known as mycotoxins.

The present invention further comprises the use of both the diet and antifungal substances either as the sole therapies for the diabetes or in combination with conventional chemotherapeutical, pharmaceutical, surgical, immune-modulating, vaccine-related, or any combination of conventional therapies.

In addition, the present invention comprises the use of the specific diet, with or without antifungal substances, if both or either are used in conjunction with any alternative type of therapy. Alternative therapies are those that are currently defined by the National Institutes of Health's Office of Alternative Medicine and/or the National Center for Complimentary and Alternative Medicine. Such alternative practices may include nutrition, massage, chiropractic manipulation, mind-body medicine, Ayurveda, naturopathy, homeopathy, reflexology, magnet therapies, hypnosis, vitamin and herbal therapies, biofeedback, osteopathic manipulation therapy, aromatherapy, and others.

In addition, the present invention applies to the use of antifungals, with or without the specific diet, to any disease or syndrome characterized in part by insulin resistance and/or hyperglycemia, with or without damage to the pancreas, whether the condition can be related to being caused by diet and lifestyle or caused by pharmaceutical or naturally-occurring chemicals or infectious organisms.

In addition, the present invention applies to the use of the specific diet, with or without antifungal substances, in combination or singularly, in the treatment and/or prevention of the co-morbidities related to diabetes. These conditions include: hypertension, cerebrovascular disease, atherosclerotic coronary artery disease, macular degeneration, diabetic retinopathy (eye disease) and blindness, cataracts—systemic inflammation (characterized by elevation of inflammatory markers such as erythrocyte sedimentation rate or C-reactive protein), birth defects, pregnancy related diabetes, pre-ecclampsia and hypertension in pregnancy, kidney disease (renal insufficiency, renal failure etc.), nerve disease (diabetic neuropathy), superficial and systemic fungal infections, congestive heart failure, gout/hyperuricemia, obesity, hypertriglyceridemia, hypercholesterolemia, fatty liver disease (non-alcoholic steatohepatitis, or NASH), and diabetes-related skin diseases such as Necrobiosis Lipoidica Diabeticorum (NLD), Blisters of diabetes (Bullosis Diabeticorum), Eruptive Xanthomatosis, Digital Sclerosis, Disseminated Granuloma Annulare, and Acanthosis Nigricans.

In addition, the present invention applies to the treatment and prevention of such diabetes precursors as “Syndrome X,” also known as Metabolic Syndrome, as well as Impaired Glucose Tolerance. Metabolic Syndrome is currently defined as having 3 out of the 5 following conditions:
Abdominal obesity (waist circumference greater than 40 inches in men or greater than 35 inches in women)
Hypertriglyceridemia (triglyceride level greater than or equal to 150mg/dl)
Low HDL-Cholesterol (greater than 40 mg/dl in men or greater than 50 mg/dl in women)
High blood pressure (greater than or equal to 130/85)
High fasting glucose (impaired glucose tolerance (IGT): fasting blood sugar between 110 and 126 mg/dl) See, Blackburn, G, et al. The Obesity Epidemic:

Prevention and Treatment of the Metabolic Syndrome. Medscape.com. Released Sep. 18, 2002.

Impaired glucose is defined as having a fasting blood sugar of between 110 mg/dl and 125 mg/dl, or by having an abnormal 3 hour glucose tolerance test.

In addition, the present invention applies to the use of the specific diet, with or without antifungal substances, in combination or singularly, in the prevention and treatment, whether complementary to or in place of traditional medical therapies, of multiple sclerosis, another disease of unknown etiology.

### DETAILED DESCRIPTION

Diabetes and Fungal Infections

It has long been recognized that fungal and yeast infections are more common in a diabetic person. In fact, in the past scientists were able to identify those at risk for diabetes by noting their propensity to suffer from recurrent yeast and fungal infections. (Wilson, J W, Plunkett, O A., The Fungous Diseases of Man, University of California Press., Berkeley, Los Angeles, and London 1970). However, conventional teachings still maintain that diabetes creates the predisposition for fungal infections. Based on the available scientific literature, the fungus—or a fungal metabolite—is actually the predisposing factor for the development of diabetes.

An in-depth look at the diabetes epidemic, fungi, and mycotoxins is offered in the book Infectious Diabetes. (Kaufmann, D., MediaTrition, Inc. 2003).

The History of Diabetes and Fungus

(Dr. A. V. Costantini's 1994 lecture in Canada)

1788 Whytt: Observed that diabetes mellitus (DM) and gout go hand in hand.

Observation: Polynesians have a very high rate of diabetes, gout, obesity and atherosclerosis. The Polynesians are known to consume large quantities of a product called “Poi.” Some of the men consume 10-20 pounds of Poi per day. Poi is a fermented fruit concoction consisting of yeast-fermented bananas and breadfruit.

1954 Griffits: Uric acid produces DM in animals.

1963 Svlhia: Sacchromyces yeast produce uric acid.

1990 Coleman: Mice fed a 10% brewer's yeast diet developed DM.

1976 Isogai (Tokyo, Japan): Cryptococcus fungi were found in the islets of Langerhans cells (pancreas) in two children who died from DM. The researchers in later studies injected Cryptococcus into the pancreatic artery of laboratory animals and induced necrosis of the islets of langerhans. Cryptococcus is known to produce alloxan.

1980 Pojo: Alloxan, a uric acid metabolite, injures insulin-producing beta cells in the islets of Langerhans of the pancreas.

1990 Chase: Type I DM could be cured if treated with cyclosporin A. within four months of onset of the disease.

1990 Moody: Cyclosporin A, a fungal poison and pharmaceutical drug, is antifungal against Cryptococcus.

1981 Hayes: Streptozotocin induces experimental DM in animals. Hayes points out that Streptozotocin is a mycotoxin produced by Streptomyces achromogenes mold.

1981 Helgason: ingestion of cured mutton, a holiday dish, by Icelandic women at the time of conception caused DM in their offspring.

1973 Esher: Cured mutton contains ochratoxin, sterigmatocystin, patulin, and penicillic acid—all fungal mycotoxins.

1990 Raha: L-asparaginase (fungally produced) induces DM in experimental animals.

Recent Findings Associating Diet and Fungal Toxins with Diabetes

Two separate studies, one in the United States and the other in Germany, conclude that feeding infants cereals early in life significantly increases the infants' risk of developing type 1 diabetes later in life:
Timing of Initial Cereal Exposure in Infancy and Risk of Islet Autoimmunity—Jill M. Norris, Katherine Barriga, Georgeanna Klingensmith, Michelle Hoffman, George S. Eisenbarth, Henry A. Erlich, and Marian Rewers—JAMA. 2003;290:1713-1720.
Early Infant Feeding and Risk of Developing Type 1 Diabetes-Associated Autoantibodies—Anette-G. Ziegler, M D; Sandra Schmid, PhD; Doris Huber; Michael Hummel, M D; Ezio Bonifacio, PhD.—JAMA. 2003;290:1721-1728.
   Findings of the Studies

Context: Dietary factors modifying type 1 diabetes mellitus (DM) risk have been proposed, but little is known if they trigger the islet autoimmunity that precedes clinical disease.

Objective: To determine whether breastfeeding duration, food supplementation, or age at introduction of gluten-containing foods influences the risk of developing islet autoantibodies.

Design and Setting: Prospective natural history cohort study conducted from 1989 to 2003 in inpatient/outpatient clinics in Germany.

Participants: The BABYDIAB study follows newborn children of parents with type 1 DM. Eligibility requirements were met in 1610 children. Blood samples were obtained at birth, age 9 months, 2, 5, and 8 years. Dropout rate was 14.4% by age 5 years. Breastfeeding data were obtained by prospective questionnaires (91% complete), and food supplementation data were obtained by family interview (72% for food supplementation and 80% for age of gluten introduction)

Main Outcome Measure: Development of islet autoantibodies (insulin, glutamic acid decarboxylase, or IA-2 antibodies) in 2 consecutive blood samples.

Results: Life-table islet autoantibody frequency was 5.8% (SE, 0.6%) by age 5 years. Reduced total or exclusive breastfeeding duration did not significantly increase the risk of developing islet autoantibodies. Food supplementation with gluten-containing foods before age 3 months, however, was associated with significantly increased islet autoantibody risk (adjusted hazard ratio, 4.0; 95% confidence interval, 1.4-11.5; P=0.01 vs children who received only breast milk until age 3 months). Four of 17 children who received gluten-containing foods before age 3 months developed islet autoantibodies (life-table 5-year risk, 24%; SE, 10%). All 4 children had the high-risk DRB1*03/04,DQB1*0302 genotype.

Gluten-containing foods are such grains as wheat, rye, barley and oats. Wheat, rye, and barley are commonly-contaminated with fungal mycotoxins. (Etzel, R., Mycotoxins, Journal of the American Medical Association, 287(4): 425-427, Jan. 23/30, 2002; Council for Agricultural Science and Technology, Mycotoxins: Risks in Plant, Animal and Human Systems, Task Force Report No. 139, Ames, Iowa, January 2003).

Antibodies are immune-system protein structures that are made by the human body's B-cells of the immune system and that are directed against foreign chemicals and germs in our body. Auto-antibodies are antibodies that are directed-supposedly by mistake-against the human body's tissues, organs, and cells. Conventional medicine claims that this is an abnormal response by they human body, and the manifested condition is called an auto-immune disease. If, however, a mycotoxin that preferentially attacks the islet cell in the pancreas is able to alter that pancreatic cell (i.e., the cell is now chemically tainted), then the human body will see that cell as foreign, or at least abnormal. Hence, an immune attack against that abnormal, chemically infected cell is, in this case, a normal response by our immune system, not an abnormal, auto-immune phenomenon.

Fungal Toxin in Potato Scab Causes Type 1 Diabetes

A common toxin found in the potato scab in root vegetables is linked to type 1 diabetes:
Bafilomycin, a macrolide antibiotic (mycotoxin) made by the Streptomyces griseus mold and found in the black, scab lesions on root vegetables (especially potatoes) caused diabetes in 100% of the offspring of mother mice who were fed this toxin. (www.onenews.nzoom.com, citing a study by Paul Zimmet et al., June 2003, director of the International Diabetes Institute in Melbourne, Australia).
Bafilomycin is a heat-stable fungal toxin that cannot be destroyed in the cooking process.
   The Fungus/Mycotoxin Association with Type 2 Diabetes

Aspergillus and Penicillium fungi are common contaminants of peanuts and corn. (The Council For Agricultural Science and Technology, Mycotoxins: Risks in Platnt, Animal, and Human Systems, Task Force Report No. 139, January 2003, Ames, Iowa). They make mycotoxins such as ochratoxin, patulin, and aflatoxin.

The effects of Ochratoxin in mammals:
It prevents mammalian cells from breaking down sugar normally so that the levels of sugar in the blood remain high. It also creates insulin resistance, which leads to the high blood sugar levels seen in type 2 diabetes. (Verma, R., Shalini, M., Hyperglycemia induced in rabbits exposed to ochratoxin, Bull Environ Contam Toxicol, April 1998, 60(4):626-31; Subramanian, S., et al., Ochratoxin A toxicity on carbohydrate metabolism in rats, Bull Environ Contam Toxicol, August 1989, 43(2):180-4; Huff, W E, et al., Decreased glycogen mobilization during ochratoxicosis in broiler chickens, Appl Environ Microbiol, January 1979, 37(1) :122-6; Suzuki, S. et al., Effect of ochratoxin A on carbohydrate metabolism in rat liver, Toxicol Appl Pharmacol, April 1975, 32(1):116-22; Szczech, G M, et al., Ochratoxicosis in Beagle dogs, I. Clinical and clinicopathological features, Vet Pathol. 1973, 10(2):135-54).
* It also causes kidney damage, a very common occurrence in diabetes. (CAST 2003, Rodricks 1977).

The effects of Patulin in mammals:
A common contaminant of apple juice and processed, apple products (Council for Agricultural Science and Technology, Mycotoxins: Risks in Plant, Animal, and Human Systems, Task Force Report 139, January 2003.)
It inhibits human cells from using oxygen normally (inhibits aerobic respiration). (Rodricks, J., Mycotoxin in Human and Animal Health, Pathotox Publishers, Inc., Park Forest South, Ill., 1977., p 613).
Human cells cannot live without oxygen, but fungi with anaerobic metabolism capacities can. Thus, mycotoxin exposure can create an environment that favors fungi in the human body.

The effects of Aflatoxin B1 in mammals:
Found in mold-contaminated corn, wheat, peanuts and other grains. (Council for Agricultural Science and Technology, Mycotoxins: Risks in Plant, Animal, and Human Systems, Task Force Report 139, January 2003; Etzel, R., Mycotoxins, Journal of the American Medical Association, 287(4), Jan. 23/30, 2002).
It blocks the breakdown of sugar in the liver as well as the sugar in other cells in the human body, again resulting in high levels of blood sugar that human cells cannot use. (Cheeke, P. R., Natural Toxicants in Feeds, Forages, and Poisonous Plants, 1998, Interstate Publishers, Inc., Danville, Ill.). This, in turn, creates the perfect environment for fungi, which preferentially feed on sugars/carbohydrates.
   Other Mycotoxins and Their Association with Type 1 and Type 2 Diabetes

Streptozotocin
Aside from causing type 1 diabetes by destroying the cells in the pancreas, it also “causes insulin resistance” in human cells—the very definition of type 2 diabetes. (Samiec, P. S., et al., Glutathione in human plasma: Decline in association with aging, age-related macular degeneration, and diabetes, Free Radic. Biol. Med., Mar. 15/24(5): 699-704, 1998; Szkudelski, T., The mechanism of alloxan and streptozotocin action in B cells of the rat pancreas, Physiol res. 2001, 50(6) :537-46; Streptozotocin: CAS No. 18883-66-4, Ninth Report on Carcinogens, US Dept of HHS, Public Health Service, National Toxicology Program., Revised January 2001).

Alloxan
Alloxan is an oxidation product of uric acid. Uric acid is made by, among other organisms, fungi. (Helbig, F. et al., Uric acid is a genuine metabolite of Penicillium cyclopium and stimulates the expression of alkaloid biosynthesis in this fungus, Applied and Environmental Microbiology, April 2002. P. 1524-1533, Vol. 68, No. 4, January 2002).
Injection of animals with alloxan causes type 1 diabetes via destruction of the beta cells in the pancreas. (Wallace, A., Principles and methods of toxicology, Raven Press, New York, 1989., p. 694; Moneim, A., et al., Effects of Nigella Sativa, fish oil and gliclazide on alloxan diabetic rats 1—Biochemical and histopathological studies, J. Egypt., Ger. Soc. Zool., Vol. 23(A), 237-265, 1997).
Alloxan also causes insulin resistance, characteristic of type 2 diabetes. (Ader, M, et al., Evidence for direct action of alloxan to Induce insulin resistance at the cellular level, Diabetologia. November 1998, 41(11):1327-36).
In addition, injection of animals with alloxan can cause elevated serum cholesterol, triglyceride, and total lipid levels. (Moneim, A., et al., Effects of Nigella Sativa, fish oil and gliclazide on alloxan diabetic rats 1—Biochemical and histopathological studies, J. Egypt, Ger. Soc. Zool., Vol 23(A), 237-265, 1997; Szkudelski, T., The mechanism of alloxan and streptozotocin action in B cells of the rat pancreas, Physiol Res., 2001, 50(6):537-46).
   Oxalic Acid

Oxalic acid is a harsh chemical found in small quantities in certain plants. Aspergillus fungi, however, can liberate large quantities of oxalic acid during an infection (a so-called fungus ball) in a human lung. (Kibbler, C C., Principles and Practice of Clinical Mycology, John Wiley and Sons, Ltd., West Sussex, England, 1996).

Oxalic acid can cause death in mammals by inhibiting carbohydrate metabolism in animals. (Wallace, A., Principles and methods of toxicology, Raven Press, New York, 1989, p. 694).

Conventional and Alternative Treatment of Type 2 Diabetes: Sulfa Drugs and Over the Counter Supplements

Sulfa-based, oral diabetic drugs (glypizide, glyburide, etc.)
Per the drug information listed in Physician's Desk References, scientists are not fully sure why sulfa-based prescription diabetes medicines lower blood sugar. Their action cannot solely be explained by increasing the output of insulin.
Since diabetes is caused by fungi and their mycotoxins, sulfa drugs help lower blood sugar in type 2 diabetes in part because they are antifungal. (Large, E. C., The Advance of the Fungi, p44., Dover publications, New York, N.Y., 1962).
Chromium, Garlic, and other over-the-counter supplements found to be helpful in type 2 diabetes also have documented antifungal activity. (Costantini, A V., Fungalbionics Series. Etiology and Prevention of Atherosclerosis, Johann Friedrich Oberlin Verlag, Freiburg, Germany, ISBN 3-930939-04-5, 1996).
   The Metabolic Syndrome, Fungi and Their Associated Mycotoxins

Formerly known as “Syndrome-X,” the metabolic syndrome is not always associated with being overweight. In fact, 18% of people with the metabolic syndrome in one study were classified as having normal body weight, and 67% were obese. (Marchesini, G., et al., Nonalcoholic fatty liver, steatohepatitis, and the metabolic syndrome, Hepatology, April 2003, 37(4):917-23). Nevertheless, obesity is one of the most common findings in this condition. Other, diagnostic criteria for Metabolic Syndrome are as follows:

One must have 3 out of the following 5 conditions:
Abdominal obesity (waist circumference greater than 40 inches in men or greater than 35 inches in women).
Hypertriglyceridemia (triglyceride level greater than or equal to 150 mg/dl).
Low HDL-Cholesterol (greater than 40mg/dl in men or greater than 50 mg/dl in women).
High blood pressure (greater than or equal to 130/85).
High fasting glucose (Impaired glucose tolerance (IGT)—fasting blood sugar between 110 and 126 mg/dl) (Blackburn, G, et al., The Obesity Epidemic: Prevention and Treatment of the Metabolic Syndrome, Medscape.com, Released Sep. 18, 2002).

Pursuant to these criteria, about 47 million Americans have the Metabolic Syndrome. That means that one in four Americans are living with this cluster of signs and symptoms. The etiology of the Metabolic Syndrome has been elusive, but is currently attributed to “improper nutrition and inadequate physical activity.” (Blackburn, G, et al., The Obesity Epidemic: Prevention and Treatment of the Metabolic Syndrome, Medscape.com, Released Sep. 18, 2002). Not surprisingly, every single component of this syndrome can be reversed by weight loss. (Blackburn, G, et al., The Obesity Epidemic: Prevention and Treatment of the Metabolic Syndrome, Medscape.com, Released Sep. 18, 2002).

The obesity epidemic is flourishing in the face of our current medical-society-based and government-established food plans, thereby creating questions about the validity or appropriateness of those food plans. Naturally, food plans cannot be solely to blame for these epidemics. Therefore, it is the wrong food plan, along with a sedentary lifestyle, poor snack choices, and over-consumption of antibiotics, that is to blame for these epidemics of obesity and the metabolic syndrome.

The twofold reason why carbohydrates are the wrong food to place at the foundation of the current, popular food plans is that (1) they are easily converted into fats (Harper, et al., Review of Physiological Chemistry, 16^{th} ed., Los Altos, Calif., 1977) and, (2) they are commonly contaminated with disease-causing mycotoxins. (Etzel, R., Mycotoxins, Journal of the American Medical Association, 287(4), Jan. 23/30, 2002). In the ideal world, grains could be a safe choice for a staple food. But the facts remain that ours is an inactive society. Individuals are not burning off the grains that are consumed, and grains are a stored and processed product. Storing and processing grains (along with droughts and floods) increases the risks of mycotoxin contamination. Even the seeds used to plant new crops can be contaminated with mycotoxin-producing fungi, so planting one's own garden does not avoid the inevitable exposure to fungi in certain grain-bearing plants. This is the case with corn and corn kernels used to plant new crops. Luckily, rice and oats are less prone to fungal contamination and, as such, they are perhaps better choices for staple grains.

Fatty Liver Disease and its Association with Mycotoxins

Abnormally elevated liver enzymes or fatty deposits in the liver are not part of the criteria for diagnosing metabolic syndrome. Nevertheless, a majority of people (73%) with the metabolic syndrome have what's called nonalcoholic steatohepatitis (NASH). Whether one has the metabolic syndrome or not, if one's liver enzymes are elevated on a blood test, a doctor will be able to determine whether NASH or some other, infectious agent is responsible for the rise in liver enzymes (AST, or SGOT, and ALT, or SGPT).

Essentially, NASH describes a liver that is inflamed and full of fatty deposits, similar to what might happen if one drinks an abundance of alcohol over long periods of time. Only, in NASH, alcohol is not part of the picture. NASH can progress to severe, fatal liver disease over many years. NASH results in cirrhosis (irreversible scarring, like that seen in kidney failure) in 20-25% of patients who have it and liver-related deaths in 8-15% of patients. (Resnick, R., Chopra, S., Nonalcoholoic steatohepatits: A common hepatic disorder, Family Practice Recertification, Vol 24, No. 9., August 2002). And just as diabetes and hypertension are fueling a culture of people with kidney failure, the huge number of people with metabolic syndrome is going to give rise to a large population of people with liver failure in the next 10 or 20 years—unless a cause and, therefore, treatment, can be identified. (Marchesini, G., et al., Nonalcoholic fatty liver, steatohepatitis, and the metabolic syndrome, Hepatology, April 2003, 37(4):917-23).

The etiology of fatty liver in overweight individuals remains “yet to be determined,” though it is suspected to have something to do with insulin resistance. (Russo, M., Jacobson, I., Nonalcoholic fatty liver disease, Hospital Physician, November 2002). The etiology has already been determined. For example, aflatoxin, the Aspergillus fungal toxin, is known to cause fatty liver, hepatitis, and fibrosis (scarring) in humans and animals. (CAST, Mycotoxins: Risks in plant, animal, and human systems, Task Force Report No. 139, Jan 2003, Council for Agricultural Science and Technology, Ames, Iowa). Ochratoxin, made by Aspergillus and Penicillium fungi, also causes fatty liver in humans and animals. (Class course in Advanced Food Microbiology, Microbial foodborne pathogens, http://class.fst.ohio-state.edu/fst736/sect4.htm. June 2003; Rodricks, J., et al., Mycotoxins in Human and Animal Health. Pathotox Publishers, Inc., Park Forest South, Ill., 1977, p. 492). In addition, mycotoxins such as streptozotocin induce a state of insulin resistance. (ID TNO Animal Nutrition, Diabetic pig characterized by hepatic and cellular insulin-resistance, http://www.id.dlo.n1/ID-Lelystad/documenten/flyers/IDTNO 22.0701 koopmans uk.pdf.).

These facts are only relevant if, in fact, people were consuming mycotoxins in small quantities—as food contaminants and prescriptive antibiotics—on a regular basis. Scientists have already established that this is, indeed, the case. (CAST, Mycotoxins: Risks in plant, animal, and human systems, Task force report No. 139, Jan 2003, Council for Agricultural Science and Technology, Ames, Iowa; Etzel, R., Mycotoxins, Journal of the American Medical Association, 287 (4): 425-427, Jan. 23/30, 2002).

In addition, in a biopsy a liver with nonalcoholic-related fatty changes looks “almost identical” to that of a liver damaged by alcohol abuse. (Kichian, K., et al., Nonalcoholic fatty liver disease in patients investigated for elevated liver enzymes, Canadian Journal of Gastroenterology, January 2003, 17(1):38-42). And alcohol is but a mycotoxin made by the yeast Saccharomyces cerevisiae, i.e., brewer's yeast.

Ours is a population of people who are obediently following their grain-based dietary recommendations and taking loads of unnecessary antibiotics (most of which are fungal by-products themselves), while at the same time they are developing classic symptoms of mycotoxin exposure. But instead of calling the disease what it most likely is—a mycotoxicosis—it is called NASH, or metabolic syndrome, or any other of a dozen unknown etiology diseases.

Kidney Damage/Chronic and Acute Renal Insufficiency and Mycotoxins

Nearly a quarter of all adults over the age of 45 have some form of chronic, renal insufficiency. (Jancin, B., Chronic Renal Insufficiency Strikes 23% of Adults, Family Practice News, Jun. 1, 2002).

Folmer Elling has a full chapter dedicated to the “Morphological Aspects of Mycotoxic Nephropathy” in a book covering the ill effects of mycotoxins in humans. In it, he describes the renal-toxic effects of ochratoxin, a toxin produced by Aspergillus and Penicillium species of molds. (Rodricks, J., et al., Mycotoxins in Human and Animal Health, Proceedings of a conference on mycotoxins in human and animal health, pp. 499-506, Pathotox publishers, Park Forest South, Ill. 1977).

Ochratoxin has been documented to cause kidney damage in all animal species tested thus far. Ochratoxin is suspected to cause Endemic Nephropathy, also known as “Balkan,” or “IgA” nephropathy, a form of kidney failure seen in central Europe where ochratoxin has been found in high levels in the food supply. In one random sampling 56% of Germans had detectable levels of ochratoxin in their bloodstream. Ochratoxin, an unregulated mycotoxin in the United States, is typically found in barley, corn, wheat, oats, rye, green coffee beans, and peanuts. (Bray, G., Ryan, D., eds., Pennington Center Nutrition Series, Volume 1: Mycotoxins, Diabetes and Health, p. 42-43; Council for Agricultural Science and Technology, Mycotoxins: Economic and Health Risks, Task Force Report Number 116, p.35. November 1989, CAST, Ames, Iowa).

IgA nephropathy is the most common cause of glomerulonephritis, or kidney disease, in the world. (Council for Agricultural Science and Technology, Mycotoxins: Economic and Health Risks, Task Force Report Number 116, p.35, November 1989, CAST, Ames, Iowa). A Fusarium mold toxin known as deoxynivalenol, of the trichothecene group of toxins, causes accumulation of the antibody IgA in the filtering areas (called glomeruli) of the kidneys in mice, identical to the pathologic process seen in Balkan Nephropathy in humans. (Council for Agricultural Science and Technology, Mycotoxins: Economic and Health Risks, Task Force Report Number 116, p.35, November 1989, CAST, Ames, Iowa). The overactive immune response to the mycotoxin in the kidneys leads to permanent damage.

The trichothecene mycotoxins are 40 times more toxic when inhaled than when consumed in contaminated foods. (Peraica, M., et al., Toxic Effects of Mycotoxins in Humans, Bulletin of the World Health Organization, WHO website, 1999). One case study documented kidney failure (acute renal failure) caused by inhaled mycotoxins. A farmer developed kidney failure after she had been working in a granary containing Aspergillus ochraceus-infected wheat. The mold Aspergillus ochraceus makes ochratoxin, and wheat that is infected with this mold liberates ochratoxin into the air, which can be harmful if inhaled, especially in an enclosed area like a grain silo. The kidney biopsy on the farmer showed characteristic acute tubular necrosis (ATN) and “minimal change” lesions, which are certain tissue changes seen in the biopsy of a failed kidney. She recovered slowly and fully, needing only temporary dialysis in the hospital following avoidance of the toxin.

Because streptozotocin causes diabetes, it is interesting that exposure to this same chemical, marketed under the name Zanosar®, can lead to “severe” or even “fatal kidney disease” in humans. (Physicians' Desk Reference, 48^{th} edition, Medical Economics Data Production Company, Montvale, N.J., 1994; Ishikawa, A., et al., Mechanism of cyclosporin induced nephrotoxicity, Transpl Proc., 31:1127-1128, 1999). It is directly toxic to the kidneys and can lead to tissue damage, similar to the damage in the pancreas that leads to diabetes in experimental animals.

Cyclosporin-A, a fungal byproduct from the Tolypocladium inflatum fungus (Turner, G., Exploitation of fungal secondary metabolites old and new, Microbiology Today, Vol. 27, August, 2000) is classified as a macrolide antibiotic, although it is not used as an antimicrobial in humans. Erythromycin, clarithromycin, and azithromycin are also macrolide antibiotics. Cyclosporin-A, however, is used for the purpose of suppressing the human body's immune system so that it will not reject a foreign, transplanted organ. It is a known vasoconstrictor, or a substance that constricts blood vessels. Because of its action, almost 100% of the time, persons receiving therapeutic doses of cyclosporin-A will develop hypertension. (Cifkova, R., Haller, H., Cyclosporin-induced hypertension, European Society of Hypertension Scientific Newsletter, 2001, 2:No. 8).

Constricting blood vessels, similar to squeezing a hose, also leads to altered perfusion through the kidneys. The kidneys normally help to regulate blood pressure in the body through the action of their various hormones and fluid balance mechanisms that are put into play, in part, based on the amount of blood flow that they receive. When this blood flow is artificially altered by this mycotoxin, the kidneys, sensing a decrease in blood flow, can be stimulated to increase the blood pressure in the body. Over time, chronic oxygen and nutrient deprivation, caused by lack of blood flow, can lead to organ damage.

Nerve Damage/Neuropathy Seen in Diabetes, and its Association with Fungi/Mycotoxins

A disturbing problem that patients with long-standing diabetes often encounter is that of nerve damage. If a person has had diabetes for 10 or 20 years, then they will likely suffer from numbness, tingling, burning sensations, or pain in various parts of the body. The legs are often most frequently affected, but hands and internal organs can be affected as well. Digestion and intestinal mobility problems can occur if the nerves to the stomach or intestines are damaged. When this happens, the nerves can no longer stimulate the muscles of the intestines to move food along through the stomach and digestive tract, so intestinal blockage becomes a serious problem. Nausea and vomiting may be a symptom of this problem (called gastroparesis). Impotence can arise in males that can also happen as a result of damage to the delicate nerves of the genital area.

Other various symptoms of nerve damage may include dizziness, diarrhea or constipation, wasting of the muscles in the arms or legs, difficulty urinating, loss of balance and generalized weakness. (InteliHealth.com, Diabetic Neuropathy: The Nerve Damage of Diabetes, December 2002).

Burning feet and legs is a common complaint of people with diabetic nerve damage. This can result either from the lack of blood supply to the legs or direct damage to the nerve by a mycotoxin. Gliotoxin, a fungal poison produced by Aspergillus, Candida, Gliocladium and Penicillium fungal species, is extremely toxic to cells and nerves in very small concentrations. (Forsby, et. al., Cellular Neurotoxicology, Neurochem.su.se., 25 Nov., 2002). Fusarium and Aspergillus mold toxins called fumonisins are neurotoxic (can damage nerves) and are “universally present in corn and corn-based products.” (Etzel, R., Mycotoxins, Journal of the American Medical Association, 287(4), 425-427, Jan. 23/30, 2002). Simply put, “mycotoxins can cause nerve damage.” (Byrd, B., Food Safety: An International Public Health Issue, The International Electronic Journal of Health Education, December 2002, ISSN: 1529-1944).

Some other references to the fungal toxin-nerve damage link are as follows: the mycotoxin citreo-viridin causes nerve paralysis. Maltoryzine, an Aspergillus toxin causes muscle paralysis. Patulin (commonly found in processed apple products) causes nerve damage also. (Kemin.com, Kemin Americas, Inc., The Control of Mold and Mycotoxins in Ruminant Foods, December 2002). These studies have been done on farm and laboratory animals, but the medical literature has already documented the mycotoxin contamination of human foods. Alcohol, in its various beverage forms, is also toxic to nerves. (O'Connor, R., Alcoholic Neuropathy, www.EMedicine.com, December 2002). In people the type of nerve damage that alcohol can cause is very, very similar to that seen in diabetes: numbness primarily in the legs, muscle weakness and muscle wasting, and imbalance problems, among other things. Mycotoxins, plain and simple, damage nerves.

Cataracts, Retinopathy, Diabetes, and Fungi/Mycotoxin

Cataracts are more common and occur at an earlier age in people with diabetes. (http://www.uihealthcare.com/topics/diabetes/diab4401.html, February 2004). Diabetic retinopathy (DR) is the leading cause of preventable blindness in the United States. The reason for the development of cataracts in diabetes is felt to be the accumulation of sorbitol (a type of sugar) in the lens of the eyes, which then causes an osmotic pressure gradient, favoring the eyes, which leads to lens damage. In DR, the cause is essentially unknown. But it is known that the onset of retinopathy in diabetes parallels the onset of kidney disease. Both organs are rich in tiny, delicate blood vessels. A toxin that affects blood vessels would seem to attack the smallest vessels first. Given that mycotoxins are involved in causing type 1 and type 2 diabetes, mycotoxins are able to cause both cataracts and retinopathy as well.

In both high and low doses, zearalenone, a Fusarium mould toxin, causes retinopathy and cataracts in male and female rats. (NTP Technical Report on the Carcinogenesis Bioassay of Zearalenone, CAS No. 17924-92-4, In F344/N Rats and B6C3F1 Mice (feed study), National Toxicology Program, NIH Publication No. 83-1791, US Dept of Health and Human Services, October 1982). In addition, supplements, such as Vitamin C, that exhibit antifungal activity seem also to protect against the cataractogenesis property of mycotoxins. (Kikic, F, Trevithick, J R., Vitamin C reduces cytochalasin D cataractogenesis, Curr Eye Res, October 1995, 14(10) :943-9). Cytochalasin D, the mycotoxin studied in this case, is a by-product of the mould, Zygusporium mansonii, (Cytochalasin D, Zygusporium mansonii, A.G. Scientific, Inc., http://www.agscientific.com/Item/C1070.htm., February 2004.) and is known to produce abnormal corneal changes in human corneal tissue. (Kim, E K, et al., Corneal endothelial cytoskeletal changes in F-actin with aging, diabetes and after cytochalasin exposure., Am J Ophthalmol, Sep. 15, 1992. 114(3):329-35).

Inflammation, heart disease, atherosclerosis, diabetes, and fungi/mycotoxins: (see Infectious Diabetes, original printing, 2003. Chapter 12, pp 107-110; Chapter 15, pp 127-134)

Hypertension, diabetes, and fungi/mycotoxins: (see Infectious Diabetes, original printing, 2003; Chapter 14, pp 123-124)

Heart failure/congestive heart failure, diabetes, and fungi/mycotoxins: (see Infectious Diabetes, original printing, 2003; Chapter 135, pp 135-140)

Strokes/cerebrovascular disease, diabetes, and fungi/mycotoxins: (see Infectious Diabetes, original printing, 2003; Chapter 17, pp 141-143)

Pregnancy-related diabetes and fungi/mycotoxins: (see Infectious Diabetes, original printing, 2003; Chapter 8, pp 67-76). In addition: “Fungal toxin in potato scab causes Type 1 diabetes”

A common toxin found in the potato scab in root vegetables is linked to Type 1 diabetes:
Bafilomycin, a macrolide antibiotic (a mycotoxin) made by the Streptomyces griseus mold and found in the black, scab lesions on root vegetables (especially potatoes) caused diabetes in 100% of the offspring of mother mice who were fed this toxin. (www.onenews.nzoom.com, citing a study by Paul Zimmet et al., June 2003, director of the International Diabetes Institute in Melbourne, Australia). Bafilomycin is a heat-stable fungal toxin that cannot be destroyed in the cooking process.

Obesity, diabetes, and fungi/mycotoxins/fungal growth promoters: (see Kaufmann, D. “What Makes Bread Rise,” MediaTrition, Inc. Rockwall, Tex. 2004).

Multiple Sclerosis and Fungi/Mycotoxins

Multiple sclerosis (MS) is characterized by destruction of the protective sheath—called the myelin sheath—around nerves in the brain and the spinal cord. As a result, the transmission of nerve impulses to other nerves, muscles, and vital organs is interrupted. This impaired nerve function translates into symptoms such as difficulty in walking, abnormal “pins and needles” sensations throughout the body, pain, and loss of vision due to inflammation of the optic nerve, tremors, incoordination, paralysis, and impaired thinking and memory. (Nationalmssociety.org, September 2002). In addition, muscle wasting, bladder dysfunction, fatigue, osteoporosis, and a host of other problems can develop either directly or indirectly due to this nerve damage.

Although there is a genetic predisposition toward MS, as proven in studies of twins, only a third of those that are genetically susceptible will get MS, indicating there is still an outside factor involved. (Murray, J., Infection as a cause of multiple sclerosis: theories abound because no one knows the answer yet, Editorials, British Medical Journal, Vol 325:1128, Nov. 16, 2002).

MS is more common in those born and raised above the 37^{th} parallel (a line extending from Newport News, VA to Santa Cruz, Calif.); however, if a person moves to an area of low risk (i.e. below the 40^{th} parallel) prior to adolescence, they assume the lower risk of their new location. These last points support the idea of an environmental exposure link to the disease.

As outside causes are to blame, then Oppenheim, an early 1900's researcher, was close in his assertion that MS is caused by an environmental toxin. Other researchers of his day thought that there was a defect in the blood vessels or in the glial tissues. Pierre Marie, in the late 1800's, thought that MS was caused by an infectious agent. However, despite all of the “infection” theories that have been tested over the past 150 plus years, not one—whether bacteria, virus, Chlamydia or scrapie-like agent—has proven to be the culprit.

Mycotoxins are chemicals made by fungi. They are found in grains that have been contaminated with fungi and mold. Some mycotoxins are used for medicinal purposes. Antibiotics, such as penicillin and the cephalosporin drugs, are fungal metabolites—they are mycotoxins. Alcohol is a mycotoxin. Aflatoxin, the most carcinogenic substance on earth, is a mycotoxin.

The most food sources of these mycotoxins are peanuts, corn, alcoholic beverages, and wheat. Often, other foods such as barley, apples, sorghum and rye can be sources as well.

Some mycotoxins are produced in our body by the yeast in our intestines or vaginal tract. In one study, 3 women severely symptomatic for vaginal candidiasis were found to have vaginal fluid samples with significant levels of a mycotoxin called gliotoxin. (Shah, D. T, et al., In situ mycotoxin production by Candida albicans in women with vaginitis, Gynecol. Obstet. Invest., 1995, 39(1):67-9). Exposure to mycotoxins in the environment has several causes: ingestion, inhalation, skin contact, etc. Once inside the body these mycotoxins damage nerves.

It is known that in MS there is a loss of molecules called sphingolipids from the white matter in the central nervous system. (Harper, Review of Physiological Chemistry, 16th ed., 1977). It is not well-known that mycotoxins can actually disrupt sphingolipid biosynthesis. (Miller-Hjelle, PKD: An unrecognized emerging infectious disease? Emerging infectious diseases, 3(2):113-127, 1997, CDC). Specifically, gliotoxin, as mentioned above, can induce nerve cell death (apoptosis), albeit on a slightly larger scale.

Gliotoxin is a heat stable chemical made by Aspergillus, Candida, and other species of fungi. Not coincidentally, scientists have recovered a heat stable toxin from the cerebrospinal fluid (CSF) of MS patients. In this particular study, the scientists took the CSF from MS patients, heat-treated it to destroy any infectious germs, and then exposed it to nerve cells in a laboratory culture. What ensued was death of these nerve cells. The scientists called this heat-stable toxin “gliotoxin.”

The source of gliotoxin appears to be, again, primarily from the yeast and fungi within the human body. As such, gliotoxin is less important as an agricultural scourge than are other mycotoxins such as fumonisins, made by Fusarium and Aspergillus fungi, and the penetrim D toxin made by Penicillium crustosum. Fumonisins are a group of mycotoxins that happen to be neurotoxic as well as carcinogenic. They are “universally present in corn and corn-based products.” (Etzel, R., Mycotoxins, Journal of the American Medical Association, 287(4): 425-427, Jan. 23/30, 2002). Penitrem mycotoxins are found in things such as moldy apple products. Penetrem D can cause tremors, convulsions, limb weakness, and ataxis (unsteady gait), “not unlike the symptoms observed in MS.” (www.Iserloh.com/penitrem.html, July 2003).

As there are different classes of MS (chronic progressive, relapsing-remitting, etc.) it might be that the different presentations of MS are being caused by different classes of mycotoxins. In addition, the regional differences in the prevalence of MS can be explained by the particular agricultural products that dominate the most affected areas. For example, the part of America that lies above the 37^{th} parallel also happens to encompass the corn belt. As previously stated, corn is universally contaminated with mycotoxins. (Council for Agricultural Science and Technology, Mycotoxins: Risks in Plant, Animal, and Human System, Task Force Report 139, January 2003, Ames, Iowa). This area is also represented by much of the wheat belt. This is more than just a coincidence. It supports the hypothesis that exposure to an environmental toxin causes MS.

Regarding past and up-to-date treatments for MS, none of the current, conventional, pharmaceutical therapies offer a “cure.” (http://www.mercola.com/2003/mar/5/ms_drugs.htm, February 2004). In recent trials, statin drugs (cholesterol-lowering drugs) have, at least, proven effective in slowing the progression of MS. (Bouchez, C., Cholesterol drug may offer hope for MS patients, HealthScoutNews, April 2003; Edelson, E., Cholesterol drugs may treat multiple sclerosis, HealthScoutNews, Oct. 7, 2002; Verrengia, J., Statin drugs show M.S. promise, Associated press, Yahoo News, Nov. 7, 2002). Their effectiveness is not surprising, in light of the fungal/mycotoxin theory, because it is also known that statin drugs are antifungal. (Costantini, A. V., Fungalbionics Series: Etiology and Prevention of Atherosclerosis, Johann Freidrich Oberlin Verlag, Freiburg, Germany, 1998/99).

It is also known that Vitamin D as well as sunlight can reduce mortality from and positively influence the immune system in MS. (http://www.mercola.com/2000/may/28/sunlight_m_s.htm; http://www.mercola.com/2001/apr/25/vitamin_d.htm, February 2004). Other researchers have explained that the reason why these work is, once again, Vitamin D, whether taken in the form of a cod liver oil supplement or made naturally by our body from sunlight exposure, is an anti-mycotoxin. (Costantini, A. V., Fungalbionics Series: Etiology and Prevention of Atherosclerosis, Johann Freidrich Oberlin Verlag, Freiburg, Germany, 1998/99).

Finally, in regards to diet, a German researcher recently claimed that eating smoked sausage in childhood was responsible for causing multiple sclerosis later in life. (Murphy, D., German researcher claims smoked sausage linked to multiple sclerosis, Meatingplace.com, September 2002). Dr. A. V. Costantini, retired head of the World Health Organization's collaborating center for mycotoxins in food, has explained that smoked and aged meats are very often contaminated with mycotoxins (Costantini, A., et al., Prevention of Breast Cancer: Hope at Last. Fungalbionic series, Freiburg, Germany, 1998). Many times this is due to the addition of fungally-contaminated spices in the meat. (Aziz, N. Mex., Youssef, Y A, Occurrence of aflatoxins and aflatoxin-producing moulds in fresh and processed meat in Egypt, Food Addit Contam, May-June 1991, 8(3):321-31). Thus the cause of MS, according to these and other researchers, is food-related.

In another study, mice with an MS-like condition exhibited fewer symptoms and decreased progression of the illness when they were starved of their regular food rations. (http://www.mercola.com/2003/feb/12/starvation diet.htm, February 2004). Starvation works because fewer foods taken in allow fewer mycotoxins to enter the body. Following the standard food pyramid, which is a grain-based American diet, people consume on average between 0.15 to 0.5 mg of aflatoxin per day. (Etzel, R., Mycotoxins, Journal of the American Medical Association, 287(4):425-427, Jan. 23/30, 2002). Aflatoxin is the only regulated mycotoxin in America, so the level of exposure people have to the other, known mycotoxins in the diet disclosed herein is, at best, a guess. Thus, starvation or calorie-restricting diets not only deprive people of calories, but also deprive people of disease-causing, carcinogenic mycotoxins.

As mycotoxins cause MS, there are a number of steps one must take to minimize exposure to fungi and their mycotoxins. A low carbohydrate diet must be followed. Since mycotoxins are commonly found in grain foods, (Council for Agricultural Science and Technology, Mycotoxins: Risks in Plant, Animal, and Human Systems, Task Force Report 139, January 2003, Ames, Iowa; Etzel, R., Mycotoxins, Journal of the American Medical Association, 287(4) :425-427, Jan. 23/30, 2002), it would be wise to minimize grains in one's diet. Secondly, people should minimize exposure to antibiotics. Antibiotics are, for the most part, derived from fungi and are therefore classified as mycotoxins. And, antibiotics are a leading risk factor for the development of secondary (iatrogenic) fungal infections. (Kibbler, C C., Principles and Practice of Clinical Mycology, John Wiley and Sons, Ltd., West Sussex, England, 1996). Lastly, if one has any obvious signs of a fungal infection in or on one's body—quite possibly, simply having MS might qualify as an obvious sign (gliotoxin can be made by fungi and yeast that are already in the body, not necessarily by fungi that reside in contaminated foods)—one should take natural or prescriptive antifungals 5 for a period of time.

Example of a Low Carbohydrate/Low Mycotoxin/Anti-Fungal Diet

The initial phase diet (IDP)

| | Foods that are | Foods that are |
|---|---|---|
| | ALLOWED | EXCLUDED |
| Food Groups | in the diet: | from the diet: |
| 1. Sugar | None (1) | All sugars should |
| | | be excluded |
| 2. Artificial or | Stevia, Stevia Plus | Aspartame, |
| herbal sweeteners | | saccharin |
| 3. Fruit | Green apples, | All others, |
| | berries, avocados | including fruit |
| | grapefruit, lemons, | juice |
| | limes | |
| 4. Meat | Fish, poultry, beef, | Breaded meats |
| | etc. (2) | |
| 5. Eggs | Yes, all eggs are | Egg substitutes |
| | allowed | should be |
| | | avoided |
| 6. Dairy Products (3) | Yogurt (especially | All others, |
| | goat yogurt), | including |
| | cream cheese, | margarine |
| | unsweetened | and any butter |
| | whipping cream, sour | substitute |
| | cream | |
| | made with real | |
| | cream, butter | |
| 7. Vegetables | Most fresh, | Potatoes, legumes |
| | unblemished | (beans and |
| | vegetables and | peas) |
| | freshly-made | |
| | vegetable juice (4) | |
| 8. Beverages | Bottled or filtered | Coffee and tea |
| | water, non- | (including decaf) |
| | fruity herb teas, | Sodas (including |
| | fresh lemonade | diet sodas) |
| | or limeade sweetened | |
| | with | |
| | Stevia | |
| 9. Grains | No grains are | Pasta, rice, corn, |
| | allowed | wheat, quinoa, |
| | on the IPD | amaranth, millet, |
| | | buckwheat, |
| | | oats, barley |
| 10. Yeast products | No yeast products | All are excluded, |
| | are allowed | including |
| | on the IPD | bread, mushrooms, |
| | | pastries, |
| | | and alcoholic |
| | | beverages |
| 11. Vinegars | Unpasteurized apple | Pickles, salad |
| | cider vinegar, | dressings (5), |
| | black olives not | green olives, soy |
| | aged in vinegar | sauce. |
| 12. Oils | Olive, grape seed, | Partially- |
| | flax seed, etc. | hydrogenated |
| | Use cold-pressed | (“trans”) |
| | when available | oils, corn and |
| | | peanut oil |
| 13. Nuts | Raw nuts, including | Peanuts (along |
| | pecans, | with ALL peanut |
| | almonds, walnuts, | products) and |
| | cashews, | pistachios |
| | pumpkin seeds, | are excluded. |
| | sunflower seeds, | |
| | etc. | |
| (1) Honey may occasionally and sparingly be used as a sweetener if needed. | | |
| (2) Meat and fish are better if not corn-fed. This means avoiding farm-raised fish and meat, even if they are “organic.” Grass-fed beef is ideal. | | |
| (3) Dairy products are better if from range-fed cattle and animals not injected with antibiotics, hormones, or steroids nor fed silo-stored grains. Good | | |
| # products: Brown Cow, Monarch Hills, Redwood Hills. Whipping cream is liquid, unsweetened heavy cream. | | |

(4) Organically grown vegetables are preferable.
(5) Excluded because many of them are fermented products

An Example of One Week on the Initial Phase Diet

This weeklong example on this diet is not meant to be followed verbatim, and rarely is the duration limited to just one week. Rather, the following is merely to serve as an example.

MONDAY

| | |
|---|---|
| Breakfast: | Fried eggs, uncured bacon, ½ |
| | grapefruit |
| Snack: | Almonds, water (always bottled or |
| | filtered) |
| Lunch: | Tuna with celery. Herbal tea. |
| Snack: | carrot sticks, water |
| Dinner: | Steak, steamed veggies, sparkling lime |
| | water |
| (optional) Dessert: | Plain yogurt with raspberries |

TUESDAY

| | |
|---|---|
| Breakfast: | Omelet with onions, leeks, parsley, |
| | and chopped bacon |
| Snack: | celery sticks, water |
| Lunch: | Chicken salad with Phase I dressing |
| Snack: | cashews, water |
| Dinner: | Salmon fillets with lemon and butter, |
| | avocado salad |
| (optional) Dessert: | green apple |

WEDNESDAY

| | |
|---|---|
| Breakfast: | Poached eggs, freshly squeezed |
| | carrot juice |
| Snack: | walnuts, water |
| Lunch: | broccoli chicken without rice, |
| | herbal tea |
| Snack: | grapefruit, water |
| Dinner: | Steak, spinach salad with lemon juice and |
| | olive oil dressing |
| (optional) Dessert: | plain yogurt with chopped pecans and |
| | fresh cranberries |

THURSDAY

| | |
|---|---|
| Breakfast: | scrambled eggs with breakfast steak |
| Snack: | green apple wedges, almonds, herbal |
| | tea |
| Lunch: | tuna salad with lettuce |
| Snack: | broccoli, cauliflower, water |
| Dinner: | halibut with sauteed vegetables |
| (optional) Dessert: | yogurt with fresh blueberries |

FRIDAY

| | |
|---|---|
| Breakfast: | freshly squeezed carrot juice, hard |
| | boiled eggs |
| Snack: | celery sticks or green apple wedges |
| | with almond or cashew butter |
| Lunch: | beef patties, steamed and buttered |
| | asparagus |
| Snack: | sunflower seeds, water |
| Dinner: | Kaufmann's favorite meal (see recipes) |
| (optional) Dessert: | ½ grapefruit |

SATURDAY

| | |
|---|---|
| Breakfast: | Omelet with green onions, bacon, |
| | spinach leaves |
| Snack: | carrot sticks |
| Lunch: | Cucumber salad with onions, |
| | tomatoes, black olives, olive oil |
| Snack: | pecans, yogurt with blackberries, |
| | water |
| Dinner: | Steak with steamed broccoli |
| (optional) Dessert: | sauteed green apples and cranberries |
| | with roasted pecans and whipping cream |

SUNDAY

| | |
|---|---|
| Breakfast: | Freshly squeezed carrot juice, ½ |
| | grapefruit, poached eggs |
| Snack: | pumpkin seeds, water |
| Lunch: | salad with grilled tuna, herbal tea |
| Snack: | celery sticks, water |
| Dinner: | Stir-fried chicken, broccoli, snow peas, |
| | squash with butter |
| (optional) Dessert: | almonds, chamomile tea |

### ANTIFUNGAL EXAMPLES

Examples of antifungal prescriptive medications as well as naturally-occurring antifungal and anti-mycotoxin supplements to be used either alone or in conjunction with a carbohydrate-sparring diet in the treatment of a bloodstream or soft-tissue diabetes.
1. Fluconazole (Diflucan®, Apo-Fluconazole®) 200 mg in tablet or suspension form (10 mg/ml or 40 mg/ml) by mouth every other day for 30 days
2. Fluconazole (Diflucan®) 200-400 mg in tablet or suspension form (10 mg/ml or 40 mg/ml) by mouth or intravenously daily for 14 days
3. Fluconazole (Diflucan®) 200 mg in tablet or suspension form (10 mg/ml or 40 mg/ml) by mouth daily for three consecutive days, followed by 200 mg each Monday and Thursday thereafter for one month total
4. Fluconazole (Diflucan®) in any of the combinations listed in #1-3 above in combination and simultaneous with Nystatin (Mycostatin®) oral tablets, 500,000 units per tablet, 2 tablets twice a day for 30 days, or in combination with and simultaneous with any of the preparations of Nystatin listed below in #9-13.
5. Fluconazole (Diflucan®) 800 mg per day in tablet or suspension form (10 mg/ml or 40 mg/ml) intravenously for 7 days.
6. Fluconazole (Diflucan®) 200 mg by mouth in tablet or suspension form (10 mg/ml or 40 mg/ml) on day one, then 100 mg per day for the next 14 days.
7. Fluconazole (Diflucan®). 400 mg in tablet or suspension form (10 mg/ml or 40 mg/ml) by mouth daily for 3-12 months.
8. Fluconazole (Diflucan®) 400 mg in tablet or suspension form (10 mg/ml or 40 mg/ml) by mouth daily for 8 weeks.
9. Nystatin (Mycostatin®) oral tablets, 500,000 units per tablet, 2-3 tablets by mouth 2-4 times a day for 30 days, taken alone or in combination with a systemic antifungal agent.
10. Nystatin (Mycostatin®, Bio-statin®, Nystat-Rx®, Nystop®, Pedi-dri®, Nilstat®) oral suspension, 100,000 units per ml concentration, 2 cc by mouth twice a day for 14 days, taken alone or in combination with a systemic antifungal agent.
11. Nystatin (Mycostatin®, Bio-statin®, Nystat-Rx®, Nystop®, Pedi-dri®) oral suspension, 100,000 units per ml concentration, 1 cc in each side of the mouth four times a day for 10 days, taken alone or in combination with a systemic antifungal agent.
12. Nystatin (Mycostatin®, Bio-statin®, Nystat-Rx®, Nystop®, Pedi-dri®) oral suspension, 100,000 units per ml concentration, 5 cc by mouth, swished in the mouth and swallowed for 10 days, taken alone or in combination with a systemic antifungal agent.
13. Nystatin compounded powder, 500,000 units per ⅛ tsp, mixed in ½ cup of water and taken by mouth 4 times a day for 30 days, taken alone or in combination with a systemic antifungal agent.
14. Itraconazole (Sporanox®) in any of the following doses and/or regimens, alone or in combination with any of the Nystatin preparations listed in #9-13 above:
   a. 100 mg capsule or oral solution (10 mg/ml concentration) by mouth daily for 30 days
   b. 100 mg capsule or oral solution (10 mg/ml concentration) by mouth every other day for 30 days.
   c. 200 mg in capsule form or 200 mg of the oral solution (10 mg/ml concentration) by mouth twice a day for one week of each month for three consecutive months.
   d. Any of the above regimens (a-c) above preceded by:
      i. a loading dose of 200 mg intravenously twice a day for four consecutive doses, or
      ii. 200 mg, either in capsule or oral solution (10 mg/ml) form by mouth, three times a day for 3 consecutive days.
   e. 200 mg intravenously twice a day for four consecutive days, followed by 200 mg intravenously, daily for 14 days.
   f. 200 mg per day in capsule or oral solution (10 mg/ml concentration) form by mouth for 3 months.
   g. 200 mg per day in capsule or oral solution (10 mg/ml concentration) form by mouth for 6 months.
   h. 200 mg per day in capsule or oral solution (10 mg/ml concentration) form by mouth for 9 months
   i. 300 mg by mouth in capsule or oral solution (10 mg/ml concentration) form, twice a day for three days, followed by 200 mg twice a day for 12 weeks.
15. Terbinafine (Lamisil®, Apo-Terbinafine®, Gen-Terbinafine®, Novo-Terbinafine®, PMS-Terbinafine®) in any of the following doses, alone or in combination and simultaneously with any of the nystatin regimens in #9-13 above:
   a. 250 mg tablet by mouth daily for 6 weeks
   b. 250 mg tablet by mouth daily for 12 weeks
   c. 250 mg tablet by mouth, twice a day for 3 weeks
   d. 250 mg tablet by mouth daily for 2-8 weeks.
   e. 250-500 mg by mouth daily for up to 16 months.
   f. For children:
      i. 67.5 mg by mouth per day for 2-8 weeks for children weighing under 20 kg
      ii. 125 mg by mouth per day for 2-8 weeks for children weighing from 20-40 kg
      iii. 250 mg by mouth per day for children weighing over 40 kg.
   g. 250 mg tablet by mouth every other day for 30 days.
16. Ketoconazole (Nizoral®, Apo-ketoconazole®, Ketoderm®, Novo-ketoconazole®) in the following doses and/or regimens, alone or in combination and simultaneously with any of the nystatin regimens in #9-13 above:
   a. 200-400 mg by mouth daily for 2 weeks
   b.200-400 mg by mouth daily for 30 days
   c. 200-400 mg by mouth daily for 6 months.
   d. For children over 2: 3.3-6.6 mg/kg/day for anywhere from 1 week up to 6 months.
17. Clotrimazole (Mycelex®, Canesten®) 10 mg oral troche dissolved on tongue and swallowed 5 times a day for 14 days.
18. Caspofungin Acetate (Cancidas®): 70 mg loading dose intravenously on day 1, followed by 50 mg intravenously daily until the clinical status of the patient improves; taken alone or in combination and simultaneously with any of the nystatin regimens in #9-13 above.
19. Voriconazole (Vfend®): for children over 12 and adults—6 mg/kg intravenously every 12 hours for 2 doses, followed by 4 mg/kg intravenously every 12 hours until the clinical status of the patient improves, at which time the oral form of the medication—400 mg every 12 hours—is used in place of the intravenous form; taken alone or in combination and simultaneously with any of the nystatin regimens in #9-13 above.
20. Amphotericin B (ABLC®, Amphotec®, AmBisome®, ABCD®, Amphocil®, Fungizone®) in the any of the following doses and regimens, alone or in combination and simultaneously with any of the nystatin regimens in #9-13 above:
   a. 1 mg/kg/day intravenously for 14 days
   b. 0.5 mg/kg/day intravenously to a total dose of over 1500 mg.
   c. 0.5 mg/kg/day intravenously to a total dose of 5-7 mg/kg
   d. 0.5 mg/kg/day intravenously until clinical improvement is noted
   e. 0.5-1.0 mg/kg/day intravenously for 7 days
   f. 1 cc (100 mg) of the oral suspension form by mouth 4 times a day for 14 days.
21. Flucytosine (Ancobon®): 100 mg/kg/day by mouth every 6 hours until clinical improvement is noted in the patient; alone or in combination and simultaneously with any of the nystatin regimens in #9-13 above
22. Griseofulvin (Fulvicin®, Fulvicin-U/F®, Grifulvin-V®, Gris-PEG®) in any of the following doses and/or regimens, alone or in combination and simultaneously with any of the nystatin regimens in #9-13 above:
   a. 500-1000 mg per day of the microsized formula orally for ½ to 6 months
   b. 330-375 mg/day of the ultramicrosized formula orally for ½ to 6 months
   c. For children:
      i. 10-15 mg of the microsized formula/kg body weight/day for ½ to 6 months
      ii. 5.5-7.3 mg of the ultramicrosized formula/kg/day for ½ to 6 months
23. “Natural” Antifungals:
   a. Grapefruit seed extract: Citricidal® 33%—15 drops mixed in water, taken orally twice a day
   b. Olive leaf extract, 900 mg twice a day for 30 days or until clinical improvement is noted
   c. Garlic 1,000 mg fresh extract three times a day until clinical improvement is noted.
   d. Burdock root (Arctium lappa): 1,000 mg daily until clinical improvement is noted
   e. Caprylic Acid: 1500 mg three times a day until clinical improvement is noted.
   f. Pau d'arco (Tabebuia impetiginosa): 1000 mg by mouth, three times a day until clinical improvement is noted.
   g. Undecylenic acid: 250 mg three times a day until clinical improvement is noted.
   h. Selenium: 200 mcg per day by mouth as an adjunct to a carbohydrate-sparing diet (see “Initial Phase Diet,” above) and either natural or prescriptive antifungals.
   i. Zinc picolinate or zinc citrate: 30 mg daily by mouth as an adjunct to a carbohydrate-sparing diet and either natural or prescriptive antifungals.
   j. Iodine (in this case, the prescriptive form: Potassium Iodide (SSKI®, Iosat®, Pima®, Lugol's solution, KI): 5 drops three times a day by mouth, increasing to 40-50 drops 3 times a day and continuing for 3-6 months, alone or as an adjunct to a carbohydrate-sparing diet and/or either natural or prescriptive antifungals.
   k. Vitamin C, 2,000 mg per day by mouth as an adjunct to a carbohydrate-sparing diet and either natural or prescriptive antifungals.
   l. Vitamin E, 400IU twice a day by mouth as an adjunct to a carbohydrate-sparing diet and either natural or prescriptive antifungals.
   m. Vitamin D, 400 IU daily by mouth as an adjunct to a carbohydrate-sparing diet and either natural or prescriptive antifungals.
   n. Broccoli sprouts (containing sulfurophane), 250 mg capsule three times a day orally, as an adjunct to a carbohydrate-sparing diet and either natural or prescriptive antifungals.
   o. Oregano oil, in liquid extract or capsules: 15-45 mg of carvacrol (active constituent) three times a day orally, alone or as an adjunct to a carbohydrate-sparing diet and/or either natural or prescriptive antifungals.
   p. Orange Oil: 2 drops of 100% pure orange oil three times a day orally, alone or as an adjunct to a carbohydrate-sparing diet and/or either natural or prescriptive antifungals.
   q. Peppermint oil: 2 drops of 100% pure peppermint oil three times a day orally, alone or as an adjunct to a carbohydrate-sparing diet and/or either natural or prescriptive antifungals.
   r. Lemon myrtle oil (Backhousia Citriodora)—(citral is the active component): 2 drops three times a day, diluted in water, orally, alone or as an adjunct to a carbohydrate-sparing diet and/or either natural or prescriptive antifungals.
   s. Pterostilbene (in grape skin): 250 mg grape seed with grape skin extract-containing the pterostilbene-twice a day, alone or as an adjunct to a carbohydrate-sparing diet and/or either natural or prescriptive antifungals.
   t. Fenugreek (Trigonella foenum-graecum) seed: 1.22 grams three times a day, alone or as an adjunct to a carbohydrate-sparing diet and/or either natural or prescriptive antifungals.

Although preferred embodiments of the invention have been illustrated in the accompanying Drawings and described in the foregoing Detail Description, it will be understood that the invention is not limited to the embodiments disclosed but is capable of numerous rearrangements, modifications, and substitutions of parts and elements without departing from the spirit of the invention.

1. A method of treating a mammal having diabetes comprising administering to said mammal a formulation in an amount, at a frequency, and for a duration effective to reduce or eliminate said diabetes, said formulation comprising an anti-fungal agent.

2. The method of claim 1 wherein the anti-fungal agent comprises an anti-fungal agent selected from the group consisting of fluconazole, nystatin, itraconazole, terbinalfine, ketoconazole, coltrimazole, caspofungin, voriconazole, amphotericinB, flucytosine, and griseofulvin.

3. The method of claim 2 including the additional step of administering to said mammal a low carbohydrate diet.

4. The method according to claim 3 wherein the low carbohydrate diet excludes all sugars.

5. The method according to claim 3 wherein the low carbohydrate diet excludes all sugars, aspartame, and saccharin.

6. The method according to claim 3 wherein the low carbohydrate diet excludes all fruits except green apples, berries, avocadoes, grapefruit, lemons, and limes.

7. The method according to claim 3 wherein the low carbohydrate diet excludes all bread and breaded meats.

8. The method according to claim 3 wherein the low carbohydrate diet excludes all dairy products except yogurt, cream cheese, unsweetened whipping cream, sour cream, and butter.

9. The method according to claim 3 wherein the low carbohydrate diet excludes potatoes, yams, and legumes.

10. The method according to claim 3 wherein the low carbohydrate diet excludes coffee, tea, sodas, and canned juice.

11. The method according to claim 3 wherein the low carbohydrate diet excludes all grains and all food products made from grains.

12. The method according to claim 3 wherein the low carbohydrate diet excludes yeast products, including bread, pastries, mushrooms, and alcoholic beverages.

13. The method according to claim 3 wherein the low carbohydrate diet excludes pickles, salad dressings, green olives, and soy sauce.

14. The method according to claim 3 wherein the low carbohydrate diet excludes partially-hydrogenated oils, corn, and peanut oil.

15. The method according to claim 3 wherein the low carbohydrate diet excludes peanuts, all products made from peanuts and pistachios.

16. The method according to claim 3 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts, and pistachios.

17. The method of claim 1 wherein the anti-fungal agent comprises an anti-fungal agent selected from the group consisting of grapefruit seed extract, olive life extract, garlic, burdok root, caprylic acid, pau d-arco, undecylenic acid, selenium, zinc picolinate, zinc citrate, iodine, vitamin C, vitamin E, vitamin D, and broccoli sprouts.

18. The method of claim 17 including the additional step of administering to said mammal a low carbohydrate diet.

19. The method according to claim 17 wherein the low carbohydrate diet excludes all sugars.

20. The method according to claim 17 wherein the low carbohydrate diet excludes all sugars, aspartame, and saccharin.

21. The method according to claim 17 wherein the low carbohydrate diet excludes all fruits except green apples, berries, avocadoes, grapefruit, lemons, and limes.

22. The method according to claim 17 wherein the low carbohydrate diet excludes all bread and breaded meats.

23. The method according to claim 17 wherein the low carbohydrate diet excludes all dairy products except yogurt, cream cheese, unsweetened whipping cream, sour cream, and butter.

24. The method according to claim 17 wherein the low carbohydrate diet excludes potatoes, yams, and legumes.

25. The method according to claim 17 wherein the low carbohydrate diet excludes coffee, tea, sodas, and canned juice.

26. The method according to claim 17 wherein the low carbohydrate diet excludes all grains and all food products made from grains.

27. The method according to claim 17 wherein the low carbohydrate diet excludes yeast products, including bread, pastries, mushrooms, and alcoholic beverages.

28. The method according to claim 17 wherein the low carbohydrate diet excludes pickles, salad dressings, green olives, and soy sauce.

29. The method according to claim 17 wherein the low carbohydrate diet excludes partially-hydrogenated oils, corn, and peanut oil.

30. The method according to claim 17 wherein the low carbohydrate diet excludes peanuts, all products made from peanuts, all products made from peanuts, and pistachios.

31. The method according to claim 17 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts and pistachios.

32. A method of treating a mammal having diabetes indicating symptoms comprising administering to said mammal a formulation in an amount, at a frequency, and for a duration effective to reduce or eliminate said symptoms, said formulation comprising an anti-fungal agent.

33. The method of claim 32 wherein the anti-fungal agent comprises an anti-fungal agent selected from the group consisting of fluconazole, nystatin, itraconazole, terbinalfine, ketoconazole, coltrimazole, caspofungin, voriconazole, amphotericinB, flucytosine, and griseofulvin.

34. The method of claim 33 including the additional step of administering to said mammal a low carbohydrate diet.

35. The method according to claim 34 wherein the low carbohydrate diet excludes all sugars.

36. The method according to claim 34 wherein the low carbohydrate diet excludes all sugars, aspartame, and saccharin.

37. The method according to claim 34 wherein the low carbohydrate diet excludes all fruits except green apples, berries, avocadoes, grapefruit, lemons, and limes.

38. The method according to claim 34 wherein the low carbohydrate diet excludes all bread and breaded meats.

39. The method according to claim 34 wherein the low carbohydrate diet excludes all dairy products except yogurt, cream cheese, unsweetened whipping cream, sour cream, and butter.

40. The method according to claim 34 wherein the low carbohydrate diet excludes potatoes, yams, and legumes.

41. The method according to claim 34 wherein the low carbohydrate diet excludes coffee, tea, sodas, and canned juice.

42. The method according to claim 34 wherein the low carbohydrate diet excludes all grains and all food products made from grains.

43. The method according to claim 34 wherein the low carbohydrate diet excludes yeast products, including bread, pastries, mushrooms, and alcoholic beverages.

44. The method according to claim 34 wherein the low carbohydrate diet excludes pickles, salad dressings, green olives, and soy sauce.

45. The method according to claim 34 wherein the low carbohydrate diet excludes partially-hydrogenated oils, corn, and peanut oil.

46. The method according to claim 34 wherein the low carbohydrate diet excludes peanuts, all products made from peanuts and pistachios.

47. The method according to claim 34 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts, and pistachios.

48. The method of claim 32 wherein the anti-fungal agent comprises an anti-fungal agent selected from the group consisting of grapefruit seed extract, olive life extract, garlic, burdok root, caprylic acid, pau d-arco, undecylenic acid, selenium, zinc picolinate, zinc citrate, iodine, vitamin C, vitamin E, vitamin D, and broccoli sprouts.

49. The method of claim 48 including the additional step of administering to said mammal a low carbohydrate diet.

50. The method according to claim 48 wherein the low carbohydrate diet excludes all sugars.

51. The method according to claim 48 wherein the low carbohydrate diet excludes all sugars, aspartame, and saccharin.

52. The method according to claim 48 wherein the low carbohydrate diet excludes all fruits except green apples, berries, avocadoes, grapefruit, lemons, and limes.

53. The method according to claim 48 wherein the low carbohydrate diet excludes all bread and breaded meats.

54. The method according to claim 48 wherein the low carbohydrate diet excludes all dairy products except yogurt, cream cheese, unsweetened whipping cream, sour cream, and butter.

55. The method according to claim 48 wherein the low carbohydrate diet excludes potatoes, yams, and legumes.

56. The method according to claim 48 wherein the low carbohydrate diet excludes coffee, tea, sodas, and canned juice.

57. The method according to claim 48 wherein the low carbohydrate diet excludes all grains and all food products made from grains.

58. The method according to claim 48 wherein the low carbohydrate diet excludes yeast products, including bread, pastries, mushrooms, and alcoholic beverages.

59. The method according to claim 48 wherein the low carbohydrate diet excludes pickles, salad dressings, green olives, and soy sauce.

60. The method according to claim 48 wherein the low carbohydrate diet excludes partially-hydrogenated oils, corn, and peanut oil.

61. The method according to claim 48 wherein the low carbohydrate diet excludes peanuts, all products made from peanuts, all products made from peanuts, and pistachios.

62. The method according to claim 17 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts and pistachios.

63. A method of treating a mammal having multiple sclerosis comprising administering to said mammal a formulation in an amount, at a frequency, and for a duration effective to reduce or eliminate said multiple sclerosis, said formulation comprising an anti-fungal agent.

64. The method of claim 63 wherein the anti-fungal agent comprises an anti-fungal agent selected from the group consisting of fluconazole, nystatin, itraconazole, terbinalfine, ketoconazole, coltrimazole, caspofungin, voriconazole, amphotericinB, flucytosine, and griseofulvin.

65. The method of claim 64 including the additional step of administering to said mammal a low carbohydrate diet.

66. The method according to claim 65 wherein the low carbohydrate diet excludes all sugars.

67. The method according to claim 65 wherein the low carbohydrate diet excludes all sugars, aspartame, and saccharin.

68. The method according to claim 65 wherein the low carbohydrate diet excludes all fruits except green apples, berries, avocadoes, grapefruit, lemons, and limes.

69. The method according to claim 65 wherein the low carbohydrate diet excludes all bread and breaded meats.

70. The method according to claim 65 wherein the low carbohydrate diet excludes all dairy products except yogurt, cream cheese, unsweetened whipping cream, sour cream, and butter.

71. The method according to claim 65 wherein the low carbohydrate diet excludes potatoes, yams, and legumes.

72. The method according to claim 65 wherein the low carbohydrate diet excludes coffee, tea, sodas, and canned juice.

73. The method according to claim 65 wherein the low carbohydrate diet excludes all grains and all food products made from grains.

74. The method according to claim 65 wherein the low carbohydrate diet excludes yeast products, including bread, pastries, mushrooms, and alcoholic beverages.

75. The method according to claim 65 wherein the low carbohydrate diet excludes pickles, salad dressings, green olives, and soy sauce.

76. The method according to claim 65 wherein the low carbohydrate diet excludes partially-hydrogenated oils, corn, and peanut oil.

77. The method according to claim 65 wherein the low carbohydrate diet excludes peanuts, all products made from peanuts and pistachios.

78. The method according to claim 65 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts, and pistachios.

79. The method of claim 63 wherein the anti-fungal agent comprises an anti-fungal agent selected from the group consisting of grapefruit seed extract, olive life extract, garlic, burdok root, caprylic acid, pau d-arco, undecylenic acid, selenium, zinc picolinate, zinc citrate, iodine, vitamin C, vitamin E, vitamin D, and broccoli sprouts.

80. The method of claim 79 including the additional step of administering to said mammal a low carbohydrate diet.

81. The method according to claim 79 wherein the low carbohydrate diet excludes all sugars.

82. The method according to claim 79 wherein the low carbohydrate diet excludes all sugars, aspartame, and saccharin.

83. The method according to claim 79 wherein the low carbohydrate diet excludes all fruits except green apples, berries, avocadoes, grapefruit, lemons, and limes.

84. The method according to claim 79 wherein the low carbohydrate diet excludes all bread and breaded meats.

85. The method according to claim 79 wherein the low carbohydrate diet excludes all dairy products except yogurt, cream cheese, unsweetened whipping cream, sour cream, and butter.

86. The method according to claim 79 wherein the low carbohydrate diet excludes potatoes, yams, and legumes.

87. The method according to claim 79 wherein the low carbohydrate diet excludes coffee, tea, sodas, and canned juice.

88. The method according to claim 79 wherein the low carbohydrate diet excludes all grains and all food products made from grains.

89. The method according to claim 79 wherein the low carbohydrate diet excludes yeast products, including bread, pastries, mushrooms, and alcoholic beverages.

90. The method according to claim 79 wherein the low carbohydrate diet excludes pickles, salad dressings, green olives, and soy sauce.

91. The method according to claim 79 wherein the low carbohydrate diet excludes partially-hydrogenated oils, corn, and peanut oil.

92. The method according to claim 79 wherein the low carbohydrate diet excludes peanuts, all products made from peanuts, all products made from peanuts, and pistachios.

93. The method according to claim 79 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts and pistachios.

94. A method of treating a mammal having multiple sclerosis indicating symptoms comprising administering to said mammal a formulation in an amount, at a frequency, and for a duration effective to reduce or eliminate said symptoms, said formulation comprising an anti-fungal agent.

95. The method of claim 94 wherein the anti-fungal agent comprises an anti-fungal agent selected from the group consisting of fluconazole, nystatin, itraconazole, terbinalfine, ketoconazole, coltrimazole, caspofungin, voriconazole, amphotericinB, flucytosine, and griseofulvin.

96. The method of claim 95 including the additional step of administering to said mammal a low carbohydrate diet.

97. The method according to claim 96 wherein the low carbohydrate diet excludes all sugars.

98. The method according to claim 96 wherein the low carbohydrate diet excludes all sugars, aspartame, and saccharin.

99. The method according to claim 96 wherein the low carbohydrate diet excludes all fruits except green apples, berries, avocadoes, grapefruit, lemons, and limes.

100. The method according to claim 96 wherein the low carbohydrate diet excludes all bread and breaded meats.

101. The method according to claim 96 wherein the low carbohydrate diet excludes all dairy products except yogurt, cream cheese, unsweetened whipping cream, sour cream, and butter.

102. The method according to claim 96 wherein the low carbohydrate diet excludes potatoes, yams, and legumes.

103. The method according to claim 96 wherein the low carbohydrate diet excludes coffee, tea, sodas, and canned juice.

104. The method according to claim 96 wherein the low carbohydrate diet excludes all grains and all food products made from grains.

105. The method according to claim 96 wherein the low carbohydrate diet excludes yeast products, including bread, pastries, mushrooms, and alcoholic beverages.

106. The method according to claim 96 wherein the low carbohydrate diet excludes pickles, salad dressings, green olives, and soy sauce.

107. The method according to claim 96 wherein the low carbohydrate diet excludes partially-hydrogenated oils, corn, and peanut oil.

108. The method according to claim 96 wherein the low carbohydrate diet excludes peanuts, all products made from peanuts and pistachios.

109. The method according to claim 96 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts, and pistachios.

110. The method of claim 94 wherein the anti-fungal agent comprises an anti-fungal agent selected from the group consisting of grapefruit seed extract, olive life extract, garlic, burdok root, caprylic acid, pau d-arco, undecylenic acid, selenium, zinc picolinate, zinc citrate, iodine, vitamin C, vitamin E, vitamin D, and broccoli sprouts.

111. The method of claim 110 including the additional step of administering to said mammal a low carbohydrate diet.

112. The method according to claim 110 wherein the low carbohydrate diet excludes all sugars.

113. The method according to claim 110 wherein the low carbohydrate diet excludes all sugars, aspartame, and saccharin.

114. The method according to claim 110 wherein the low carbohydrate diet excludes all fruits except green apples, berries, avocadoes, grapefruit, lemons, and limes.

115. The method according to claim 110 wherein the low carbohydrate diet excludes all bread and breaded meats.

116. The method according to claim 110 wherein the low carbohydrate diet excludes all dairy products except yogurt, cream cheese, unsweetened whipping cream, sour cream, and butter.

117. The method according to claim 110 wherein the low carbohydrate diet excludes potatoes, yams, and legumes.

118. The method according to claim 110 wherein the low carbohydrate diet excludes coffee, tea, sodas, and canned juice.

119. The method according to claim 110 wherein the low carbohydrate diet excludes all grains and all food products made from grains.

120. The method according to claim 110 wherein the low carbohydrate diet excludes yeast products, including bread, pastries, mushrooms, and alcoholic beverages.

121. The method according to claim 110 wherein the low carbohydrate diet excludes pickles, salad dressings, green olives, and soy sauce.

122. The method according to claim 110 wherein the low carbohydrate diet excludes partially-hydrogenated oils, corn, and peanut oil.

123. The method according to claim 110 wherein the low carbohydrate diet excludes peanuts, all products made from peanuts, all products made from peanuts, and pistachios.

124. The method according to claim 79 wherein the low carbohydrate diet excludes all sugars, aspartame, and saccharin, all fruits except green apples, berries, avocadoes, grapefruit, lemons, and limes, all bread and breaded meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, green olives, and soy sauce, partially-hydrogenated oils, corn, peanut oil, peanuts, all products made from peanuts and pistachios.
